(19) United States
(12) Patent Application Publication
(10) Pub. No.: US 2008/0089894 A1
(43) Pub. Date: Apr. 17, 2008

(54) Transplant tolerance by costimulation blockade and T-cell activation-induced apoptosis

(76) Inventors: Terry B. Strom, Brookline, MA (US); Xin Xiao Zheng, Wallesley, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/999,412

(22) Filed: Dec. 5, 2007

Related U.S. Application Data
(63) Continuation of application No. 10/823,864, filed on Apr. 13, 2004, now abandoned.
Application No. 10/823,864 is a continuation of application No. 09/576,944, filed on May 22, 2000, now abandoned.
Application No. 09/576,944 is a continuation of application No. 09/075,311, filed on May 8, 1998, now abandoned.

(51) Int. Cl. A61K 39/395; A61K 31/436; A61P 37/06

(52) U.S. Cl. 424/144.1; 514/291

(57) ABSTRACT

The invention relates to methods of inducing tolerance and preventing transplant rejection by blocking costimulatory signals and using immunosuppressive agents, preferably rapamycin, to prevent late rejection episodes, yet not block tolerance inducing T-cell specific depletion; compositions and kits for use in such methods are also provided herein.

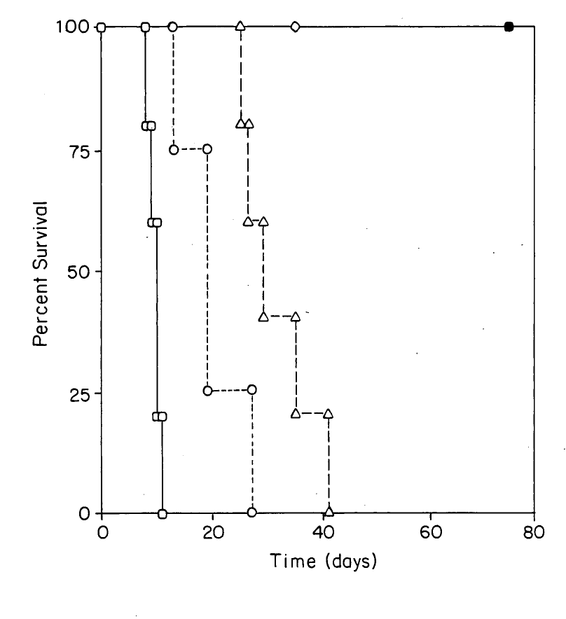

### RELATED APPLICATIONS

This application is a continuation of U.S. application Ser. No. 10/823,864, filed Apr. 13, 2004, which is a continuation of U.S. application Ser. No. 09/576,944, filed May 22, 2000, abandoned, which is a continuation of U.S. application Ser. No. 09/075,311, filed May 8, 1998, abandoned. The entire teachings of the above applications are incorporated herein by reference.

### BACKGROUND OF THE INVENTION

At present there is no established method of inducing tolerance of organ or non-hematopoietic tissue allografts in the clinic. Tolerance is necessary for drug-free, long-term allograft sustainment. The presence of an allograft causes a T-cell mediated immune response in the host. Upon activation, T-cells express interleukin-2 (IL-2) and its receptor, as well as receptors for other T-cell growth factors, leading to T-cell proliferation and differentiation into effector cells which attack the cells in the allograft.

Methods currently in use to prevent allograft rejection employ one or more of the following: 1) high doses of radiation (e.g., bone marrow allografts); and/or 2) the carefully controlled administration of immunosuppressive agents that are highly toxic. These methods have not achieved successful permanent engraftment due to a high degree of immunologic graft failure or serious permanent complications of drug toxicity, e.g. the need for kidney dialysis and the development of serious infections or malignancy from chronic immunosuppression. It would be beneficial to achieve graft tolerance and inhibit graft rejection in a significantly less toxic manner.

### SUMMARY OF THE INVENTION

The present invention relates to the treatment of mammalian graft (transplant) recipients to inhibit graft rejection and induce tolerance to grafts. More specifically, the invention relates to methods of inducing transplant tolerance by blocking T-lymphocyte costimulation signals and preventing T-lymphocyte proliferation without inhibiting normal, activation-induced T-cell (T-lymphocyte) death (e.g. T-cell depletion resulting from apoptosis). Transplant tolerance is induced by costimulation blockade and T-cell activation-induced apoptosis.

T-cell costimulation signals, (also referred to herein as costimulation signals) can be blocked by any agent that inhibits the transcription or expression of, binds to, or inhibits a protein involved in costimulation signaling. For example, such T-cell costimulation blockade agents could be any drug, antibody, biologic molecule, such as a hybrid or mutant molecule, fusion protein, or organic molecule that blocks a costimulation protein interaction. For example, such an agent could be anything that would engage (for example, associate with, or bind to) B7 (for example B7.1 (CD80), B7.2 (CD86)and B7.3), CD28, CD40, CD40 ligand (CD40L) or CTLA4. Examples of costimulation blockade agents include one or more of the following: anti-CD40 antibodies, anti-CD40L antibodies, (e.g. MRI (MR1) or 5C8), anti-B7.1 antibodies, anti-B7.2 antibodies, antibodies against shared epitopes of B7.1 and B7.2, anti-CTLA4 antibodies, CD40-Ig, CD40L-Ig and CTLA4-Ig. In one embodiment, the blockade agent would comprise a composition comprising at least one such agent, and, for example, could contain two or more such agents such as anti-CD40L and CTLA4-Ig.

The methods described herein can be used for inducing T-cell non-responsiveness to a donor tissue or organ allograft in a mammal and can inhibit rejection, thus prolonging the survival of the allograft.

Specifically encompassed by the present invention is the use of the immunosuppressive agent rapamycin to inhibit T-cell proliferation as an adjunct to costimulation blockade. As described herein, for the first time, it has been determined that rapamycin's immunosuppressive effect acts on T-cells via the IL-2 signaling pathway, resulting in the inhibition of T-cell proliferation without inhibiting T-cell activation-induced apoptosis and that the ability to block proliferation but not apoptosis is critical to enhance the beneficial attributes of costimulation blockade.

In a preferred embodiment, rapamycin (sirolimus), or a biologically active derivative thereof, is administered substantially simultaneously with the costimulation blockade agent. The immunosuppressive agent may be administered continuously. Routes of administration can include intraperitoneal, intravenous, oral or subcutaneous. Administration can be in single or multiple doses.

The invention also relates to compositions and kits for the prevention of graft rejection and the inducement of tolerance. For example, such compositions and kits can comprise one or more costimulation blockade agents and rapamycin, or a biologically active derivative or analog thereof.

### BRIEF DESCRIPTION OF THE DRAWING

The FIGURE is a graph depicting the percent survival over time (in days) of C3H/He mouse hosts receiving BALB/c mouse heart allografts or syngeneic grafts. The white squares represent the untreated hosts, the diamond represents the hosts receiving syngeneic grafts, the circles represent the hosts treated with cyclosporine, the triangles represent the hosts receiving an anti-CD40L antibody (MR1) and CTLA4-Ig with cyclosporine, and the black square represents the hosts receiving MR1 and CTLA4-Ig without cyclosporine.

### DETAILED DESCRIPTION OF THE INVENTION

Graft rejection in mammals involves a T-cell-mediated immune response. T-cells (T-lymphocytes) are activated when (1) they recognize foreign protein antigen fragments physically associated with major histocompatibility complex (MHC) molecules on the surface of antigen presenting cells (APCs) and (2) they receive costimulatory signals delivered by the APCs. Both specific ligand recognition and costimulation are required for T-cell activation.

One costimulatory signal is provided by the B7 molecules B7.1 and/or B7.2, which are structurally related costimulatory molecules on APCs. One T-cell surface molecule receptor for B7 molecules is CD28 (Tp44), a member of the immunoglobulin superfamily. Once T-cells are activated, they express CTLA4, an additional receptor for B7 cells. CTLA4 binds B7 with more affinity and avidity than does CD28. Another costimulatory signal is provided by the APC molecule CD40. This molecule binds with the T cell surface molecule CD40 ligand (CD40L), primarily on recently activated T cells.

Blockade of these costimulation signals produces potent immunosuppression. Antigen binding to the T-cell receptor in the absence of such costimulation signaling leads to a paralytic state called anergy, a loss of immune responsiveness. Anergic T-cells become refractory to activation, and cannot respond to specific antigens under optimal conditions for stimulation.

Anergy prevents T-cells from proliferating and differentiating into effector cells, even if antigen is subsequently presented by APCs. This leads, perhaps indirectly, to a state of tolerance. In other words, anergy is a stage that the host passes through before reaching a state of tolerance. As used herein, tolerance refers to the failure to reject a transplant despite the cessation of immunosuppressive therapy. Peripheral tolerance refers specifically to tolerance acquired by mature lymphocytes in the peripheral tissues.

For example, selective inhibition of T-cell costimulation using the B7-specific fusion protein CTLA4-Ig has been shown to induce allograft tolerance in some models. E.g., Olthoff et al., Nat. Med. 4(2): 194-200 (1998) and Pearson et al., Transplantation, 63(10):1463-9 (1997). Likewise, antibodies preventing interaction between CD40 and its T-cell based ligand CD40L have been shown to act synergistically with CTLA4-Ig. Further, CTLA4-Ig and CD40L-specific monoclonal antibody, 5C8, were tested in rhesus monkeys and were found to both prevent and reverse acute allograft rejection, significantly prolonging the survival of major histocompatibility complex-mismatched renal allografts in primates. Kirk et al., PNAS USA 94(16):8789-94 (1997). However, the grafts were ultimately rejected. Thus, costimulation blockade alone produces potent immunosuppression (permanent engraftment) but cannot be used as a sole treatment in transplantation because late rejection episodes occur in the most exacting pre-clinical models (nonhuman primates).

In order to advance costimulation blockade treatment into the clinic, another effective treatment modality must be added to prevent the late rejection occurring with costimulation blockade alone.

The “linchpin” of current anti-rejection regimens is the immunosuppressive drug cyclosporine (cyclosporine A or CsA). Cyclosporine is a fungal cyclic decapeptide widely used to prolong the function of transplanted organs. It exerts its immunosuppressive effects by binding to one of a group of intercellular immunophilin proteins known as cyclophilin. The cyclophilin: drug complexes then bind and inhibit the cytoplasmic serine/threonine phosphatase calcineurin. Calcineurin, which is activated by a rise of calcium ion (Ca²⁺) concentration upon T-cell antigen receptor activation, mediates activation of IL-2 (interleukin-2) transcription through its ability to dephosphorylate certain critical DNA binding proteins. Cyclosporine's binding of calcineurin thus inhibits IL-2 transcription as well as transcription of other key T-cell activation genes. IL-2 is a cytokine which stimulates T-cell proliferation and differentiation into helper T cells and cytotoxic (cytolytic) T cells (CTLs). After multiple rounds of T-cell activation, IL-2 also stimulates activation-induced apoptosis of the activated T-cells.

FK506 (tacrolimus) is a macrolide which has a similar mode of action to cyclosporine, differing primarily in that it binds to an immunophilin known as FK-binding protein (FKBP) rather than cyclophilin.

Clinical administration of cyclosporine and FK506 has been problematic. At the time of grafting, high doses are required. They are toxic to a number of organs, particularly the kidneys. This nephrotoxicity presents a serious management problem. Nephrotoxicity occurs in almost eighty percent (80%) of renal transplant patients using cyclosporine. (Kahan, B. D. Dial. Transplant. 12.620-30 (1983)). In addition to nephrotoxicity, frequent side effects of cyclosporine treatments include hypertension, hyperkalemia, hepatoxicity, and anemia. Von Graffenried, B. et al., in Cyclosporine in Autoimmune Diseases, R. Schindler, ed., Springer-Verlag, Berlin, 59-73 (1985). Progressive and possibly irreversible cyclosporine-induced deterioration of renal function has been described in heart transplant patients (Myers, B. D. et al., N. Eng. J. Med. 311:699 (1984)). Irreversible histological findings in kidneys of transplant patients given cyclosporine therapy have also been noted. Mihatsch, M. J. et al., Transplant. Proc. 15:2821 (1983); Myers, B. D. et al., N. Eng J. Med., 311:699 (1984).

Further, as shown in the Exemplification, cyclosporine blocks the tolerance-promoting properties of costimulation blockade. Whereas mouse heart allografts survived past day 70 with treatment by costimulation blockade alone, no grafts survived past day 41 when costimulation blockade was combined with cyclosporine treatment.

As a consequence of the failure to achieve permanent engraftment using cyclosporine and costimulation blockade, it has been assumed that all immunosuppressive drug therapies will inhibit the ability of costimulation blockade and other “biologics” to create tolerant T-cells.

This invention relates to the discovery that costimulation blockade allows a brief but critical period of IL-2-dependent apoptosis (programmed cell death), known as activation-induced cell death (AICD) of certain alloreactive T-cells. There is evidence that IL-2's role in this apoptosis is related to its downregulating an anti-apoptotic gene, thus mediating the interaction of the principal proteins of apoptosis, Fas and Fas ligand (FasL). The portion of alloactivated T-cells that survive IL-2-triggered apoptosis (programmed cell death) includes the “suppressor” T-cells. There is evidence that these suppressor lymphocytes are required for enduring tolerance.

Many experts believe that all immunosuppressive agents block the action or development of suppressor T-cells, and, therefore, that immunosuppressive agents cannot be administered with costimulation blockade to achieve permanent engraftment. However, cyclosporine's anti-tolerance effect is linked primarily to its inhibition of IL-2 gene expression and IL-2-dependent activation induced cell death.

This invention relates to the surprising discovery that rapamycin, another potent immunosuppressive agent, does not block the IL-2 dependent activation-induced cell death required for enduring tolerance. Like FK506, rapamycin is a macrolide which binds to the FKBP family of immunophilins. However, it has a different mode of action than cyclosporine or FK506. The rapamycin:immunophilin complex has no effect on calcineurin activity but, instead, blocks a signal transduction pathway triggered by ligation of an IL-2 receptor. The rapamycin:FKBP complex binds to TOR (“Target of Rapamycin”, the molecular target that rapamycin acts upon) and it is this interaction that inhibits growth factor signal transduction pathways. Rapamycin blocks the proliferative effects of IL-2. It also inhibits lymphocyte proliferation driven by IL-4, IL-7 and IL-15, implying a common post-receptor pathway of signaling by these cytokines. There is evidence that this pathway involves activation of a protein kinase called P70 S6K and cyclin-dependent kinase. In lymphocytes, rapamycin blocks proliferation mediated by major T-cell growth factors, e.g., IL-2, IL-4, IL-9, IL-7 and IL-15. They all use the same receptor component, the common gamma chain. Some of these factors, for example, IL-7 and IL-15, are not regulated by calcineurin, and, therefore, cyclosporine and FK506 have no effect on them. Rapamycin binds to a structure that is also common to many non-lymphocyte growth factor proliferation pathways, including the PDGF, EGF and IGF proliferation pathways. It is a powerful blocker of multiple proliferative signals at a point where their intracellular pathways tend to have common features.

Although rapamycin blocks IL-2's proliferative signals, importantly, it does not block IL-2's apoptotic signals. While IL-4, IL-7 and IL-15 share with IL-2 the capacity is stimulate T-cell proliferation, only IL-2 is necessary for T-cell activation-induced T-cell apoptosis. As shown in the Exemplification, a short course of combined rapamycin and costimulation blockade therapy provided beneficial effects for graft survival. The treatment produced tolerance to grafts and such treatment was more protective to the allografts than either costimulation blockade or rapamycin alone. Specifically, such treatment results in superior histological appearance and more rapid onset of tolerance.

Thus, this invention specifically relates to the surprising discovery that unlike cyclosporine, rapamycin, a powerful immunosuppressive, does not block the ability of costimulation blockade to cause peripheral tolerance. Therefore, while rapamycin has demonstrated activity/ability to block IL-2 stimulation intracellular signals leading to intracellular proliferation, it does not block the IL-2 signals which mediate T-cell apoptosis.

This invention encompasses methods to induce tolerance and prevent rejection using a costimulation blockade agent and an immunosuppressive agent that does not prevent selective IL-2 activation-induced T-cell death.

As used herein, the term “transplantation” refers to the process of taking a cell, tissue or organ, called a “transplant” or “graft” from one individual and placing the transplant into a (usually) different individual. The individual who provides the transplant is called the “donor” and the individual who receives the transplant is called the “host” or “recipient”. Typically, the host (i.e., the recipient of the transplant) is a mammal, such as a human. The transplant can include any transplantable cell, tissue or organ. For example, it can include a kidney, liver, heart, lung or bone marrow. A graft wherein the donor and host are genetically identical is a syngeneic graft. Where the donor and host are the same individual, the graft is called an autograft. A graft transplanted between two genetically different individuals is called an allogeneic graft (allograft). A graft transplanted between individuals of different species is called a xenogeneic graft, or a xenograft. The invention relates to all types of grafts.

As used herein, “transplant rejection” is defined as a functional and structural deterioration of the graft cell, tissue or organ due to an immune response expressed by the recipient, and independent of non-immunological causes of dysfunction. “Tolerance”, for example refers to the failure to respond to an antigen. “Peripheral tolerance” refers specifically to tolerance acquired by mature lymphocytes in the peripheral tissues.

“Protection” which can be partial or complete, refers to a state in which the effects of rejection are less than they would be if tolerance had not been induced or enhanced. The invention permits grafts and hosts to survive what would otherwise be damaging or lethal events.

By “effective amount” or “effective dose” is meant the amount of costimulation blockade or immunosuppressive agent sufficient to produce a clinically beneficial result in the treatment of animals, preferably mammals, and more preferably humans. For example, a typical effective amount of cyclosporine is in the range of 1-25 mg/day.

“Inhibition” refers to partial or complete blockade or prevention of one or more activities directly or indirectly leading to damage or rejection of a graft, or injury to a host due to an immune response to a graft.

By “normal levels of T-cell death” is meant those levels which would occur after T-cell activation and administration of a costimulation blockade agent in the absence of administration of an immunosuppressive agent.

“Costimulation” refers to secondary signaling necessary to T-cell activation, which is in addition to signaling involving interaction of antigen complex and a T-cell antigen receptor. Examples of costimulation are signaling involving interaction of B7 molecules (B7.1 and B7.2) and CD28 or CTLA4, and interaction of CD40 and CD40L. “Costimulation blockade” means partial or complete inhibition of the costimulation signaling mediating T-cell activation.

“Costimulation blockade agents” include any substance which inhibits costimulation. Such agents include any drug, protein, antibody or molecule such as a soluble ligand of a costimulation receptor such as B7, CD28, CTLA4, CD40 and CD40L, hybrid or mutant molecule or a fusion protein that blocks a costimulation protein interaction, or any substance that would inhibit, block or prevent the intracellular signaling resulting from a costimulation protein interaction. For example, such an agent could be anything that would engage (e.g., associate with, or bind to) B7 (B7.1 and B7.2), CD28, CD40, CD40 ligand (CD40L or gp39) or CTLA4. Examples of such agents include, but are not limited to anti-CD40 antibodies, anti-CD40L antibodies (e.g., MR1 (MRI) or 5C8), anti-B7 antibodies (for example, anti-B71 antibodies and anti-B72 antibodies), anti-CTLA4 antibodies, B7-Ig, CD28-Ig, CTLA4-Ig, CD40-Ig and CD40L-Ig. Costimulation blockade agents also include the extracellular domain of any of the surface proteins in soluble form, including soluble extracellular CD40, CD40L, B7, CD28 or CTLA4 domain proteins or derivatives thereof, or any substance, such as a drug, which is a receptor antagonist, or which blocks costimulation at an intracellular or extracellular level. In a preferred embodiment, the blockade comprises at least two agents, for example, anti-CD40L and CTLA4-Ig. (Kirk et al. PNAS U.S.A., 94: 8789-94 (1997)) Soluble CD40 ligands can, for example, be made by the methods disclosed in U.S. Pat. No. 5,540,926, issued Jul. 30, 1996 to Alejandro et al., and in EP 555880 issued to Aruffo et al., Aug. 18, 1993, the entire contents of which are incorporated herein by reference. Fusion proteins, including B7-Ig, CD28-Ig, CTLA4-Ig, CD40-Ig and CD40L-Ig can be made using the methods disclosed in Strom et al., WO 9631229, published Oct. 10, 1996; Linsley et al., U.S. Pat. No. 5,580,756 issued Dec. 3, 1996; Linsley et al., U.S. Pat. No. 5,521,288, issued May 28, 1996; Linsley et al., U.S. Pat. No. 5,434,131, issued Jul. 18, 1995; the entire contents of all which are incorporated herein by reference. Methods of making and using antibodies and ligands, for example anti-B7.1 antibodies and other B7.1 ligands are disclosed, for example in deBoer et al., U.S. Pat. No. 5,747,034 issued May 5, 1998 and, for example, anti-CD40 ligand antibody and soluble CD40 are disclosed in Noelle et al., U.S. Pat. No. 5,683,693, issued Nov. 4, 1997, the entire contents of both of which are herein incorporated by reference in their entirety. Costimulation blockade agents also include agents that inhibit transcription or expression of a protein which interacts with another protein to mediate costimulation.

By “rapamycin” is meant a macrolide derived from Streptomyces hygroscopicus or synthetically derived. A macrolide with immunosuppressive properties is preferred. “Rapamycin” is also intended to include biologically active rapamycin analogs and derivatives thereof (e.g. Rad). Preferred derivatives of rapamycin involve chemical modifications at the 31 and/or 42 positions of the rapamycin molecule. These modifications typically involved esterifications or etherifications. Examples of rapamycin derivatives (analogs) are included in WO 9731898 and WO 9731899, both to Guo et al., published Sep. 4, 1997; U.S. Pat. No. 5,665,772 to Cottens et al., issued Sep. 9, 1997; U.S. Pat. No. 5,677,295 to Bleyman et al., issued Oct. 14, 1997; U.S. Pat. No. 5,637,590 to Palmer et al., issued Jun. 10, 1997; U.S. Pat. No. 5,567,709 to Abou-Gharbia et al., issued Oct. 22, 1996; U.S. Pat. No. 5,563,145 to Bleyman et al., issued Oct. 8, 1996; U.S. Pat. No. 5,559,122 to Nelson et al., issued Sep. 24, 1996; and U.S. Pat. No. 5,559,120 to Abou-Gharbia et al., issued Sep. 24, 1996; the entire contents of all of which are incorporated herein by reference.

By “cyclosporine” is meant a member of a group of biologically active metabolites produced by Tolylocladium inflatum Gams (formerly, Trichoerma polysporum Rifia), and other fungi imperfecti. Especially preferred is cyclosporine A.

By “administer” is meant to introduce to an animal, preferably a human. Agents and compositions can be administered sufficiently prior to transplantation to allow for the induction of a sufficient tolerance response to provide protection against rejection, but should not be administered so far in advance of the transplant that the degree of protection is inadequate to provide the prophylactic or therapeutic effect desired. This time frame is generally from one hour to one week, depending on the organism, and the conditions of administration. In another embodiment, an agent or composition may be administered during the transplantation procedure. Immunosuppressive and costimulation agents may be administered in single or multiple doses, which may be adminsitered continuously (repeatedly).

The dosage of any of the agents administered will, of course, vary depending upon known factors such as the pharmacodynamic characteristics of the agent and any accompanying agent(s), and its mode and route of administration; the age, health, and weight of the recipient; the nature and extent of symptoms, kind of concurrent treatment, frequency of treatment, and the effect desired. One dosage is approximately 0.15 mg/kg. of the recipient. In one embodiment, one dose is administered prior to transplantation. An appropriate projected dosage for humans would be in the range of 0.15 mg/kg to 28 mg/kg (0.001 millimoles/kilogram to 0.15 millimoles/kilogram).

The particular physiological carrier in which the agent and any accompanying agent are held in solution or suspension includes, but is not limited to, water, buffered saline, polyols (e.g., glycerol, propylene glycol, liquid polyethylene glycol) and dextrose solutions. The optimum concentration of the active ingredient(s), e.g., agents, in the chosen medium can be determined empirically, according to procedures well known to those of skill in the art, and will depend on the ultimate pharmaceutical formulation desired. Methods of introduction of agents at the site of treatment include, but are not limited to, parenteral routes such as intradermal, intramuscular, intraperitoneal, intravenous, subcutaneous, intrathecal, intramedullary, and epidural; and nonparenteral routes such as transdermal, ocular, intranasal, oral, and rectal. Other suitable methods include biodegradable devices and slow release polymeric devices. In one embodiment, rapamycin or a rapamycin derivative is administered in a compound comprising a pharmaceutically acceptable salt thereof.

For parenteral administration, agents can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles can also be used. The vehicle or lyophilized powder can contain additives that maintain isotonicity (e.g., sodium chloride, mannitol) and chemical stability (e.g., buffers and preservatives). The formulation is sterilized by known techniques. Suitable pharmaceutical carriers are described in the most recent edition of Remington's Pharmaceutical Sciences, A. Osol, a standard reference text in this field of art.

Methods of administration of rapamycin and rapamycin derivatives (analogs) are disclosed in U.S. Pat. No. 5,665,728 to Gregory et al., issued Sep. 9, 1997; U.S. Pat. No. 5,646,160 to Gregory et al., issued Jul. 8, 1997; U.S. Pat. No. 5,637,590 to Palmer et al., issued Jun. 10, 1997; U.S. Pat. No. 5,563,146 to Gregory et al., issued Oct. 8, 1996; and U.S. Pat. No. 5,561,138, to Armstrong issued on Oct. 1, 1996, the entire contents of all of which are incorporated herein in their entirety.

Immunosuppressive agents are compounds that inhibit immune responses. They include steroids, such as corticosteroids, for example, prednisone; cytotoxic drugs, including azathioprine and cyclophosphamide; fungal and bacterial derivatives, including cyclosporine, FK506, and rapamycin; and antibodies, for example, antibodies to lymphocytes.

Apoptosis agents include substances that mediate programmed cell death, e.g., the programmed cell death of T-cells.

Specifically encompassed by the present invention is the use of immunosuppressive agent, e.g., rapamycin, or a biologically active derivative thereof, in conjunction with costimulation blockade agents.

In one embodiment, in addition to rapamycin, the methods of the present invention comprise the use (e.g., administration) of an immunosuppressive agent which does not interfere, inhibit or block the costimulation blockade's tolerance-mediating properties.

The immunosuppressive agent can be administered in a composition or alone prior to, simultaneously (or substantially simultaneously) with, or after, administration of a costimulation blockade agent. For example, a costimulation blockade agent, such as a B7 antibody, can be administered intravenously at the time of administration at one time or in multiple doses. The doses can occur for example, one to two weeks after transplantation. Alternatively, an immunosuppressive agent can be administered in a composition comprising at least one costimulation blockade agent.

Additionally, optionally, one or more agonistic or antigonistic agents can be included in the compositions described herein, or can be administered before, during, or after administration of costimulation blockade agent or an immunosuppressive agent. An agonist or antagonist agent is an agent e.g., which enhances or prolongs the activity of an immunosuppressive or costimulation blockade agent, or one or more agents which aid in the uptake of an immunosuppressive or a costimulation blockade agent.

Agent “antagonists” are defined herein as drugs, molecules or proteins which decrease or inhibit (including competitive inhibition) one or more biological activities of an agent. Agent “agonists” are defined herein as drugs, molecules or proteins which increase or activate one or more biological properties of an agent. Agonists and antagonists of the present invention can be administered either as individual therapeutic agents or in a composition with other therapeutic agents. Agent agonists and antagonists, together, are considered modulators. They can be administered alone, but are generally administered in a composition with a physiologically compatible pharmaceutical carrier selected on the basis of the chosen route of administration and standard pharmaceutical practice.

The compositions can also include fish oil. By “fish oil” is meant any oil derived from fish or from other forms of marine life, but preferably from fish. Preferred oils are cod liver oil and mackerel oil. Preferred is fish oil containing fatty acids of predominately the omega-3 family. By “omega-3” is meant a polyunsaturated fatty acid having the first double bond three carbons from the methyl end of the fatty acid. Compositions comprising fish oil can be used, for example, according to the methods disclosed in U.S. Pat. No. 5,118,493 to Kelley et al., issued Jun. 2, 1992, the entire contents of which are incorporated herein by reference.

“Derivatives” and “variants” of agents are agents which have been modified. They can, for example, include agents which have been modified by alterations in the amino acid sequence associated with the portions. They also include, but are not limited to, truncated and hybrid forms of agents. “Truncated” forms are shorter versions of agents. “Hybrid” forms are agents that are composed of portions of two or more agents, i.e., portions of one agent combined with portions of one or more other agents.

The methods and compositions disclosed herein can also be used for the treatment and prevention of T-cell dependent autoimmune disease, e.g., Type 1 diabetes mellitus, multiple sclerosis, psoriasis, rheumatoid arthritis, and systemic lupus erythematosus.

The present invention will now be illustrated by the following exemplification, which are not intended to be limiting in any way.

### Exemplification

### Effects of CSA, Rapamycin and Co-Stimulation Blockade on Graft Survival

Materials and methods:

BALB/c mouse heart allografts were transplanted into C3H/He mice (Jackson Labs, Bar Harbor, Me.) with strong histocompatibility barriers, which were divided into six treatment groups of 5 mice each: treated with only costimulation blockade: anti-CD40 ligand monoclonal antibody (MR1, gift of Dr. M. Sayegh, Brigham and Women's Hospital, Boston, Mass.) and CTLA4-Ig (Steiner et al., J. Immunology (1995)); treated with the costimulation blockade plus cyclosporine (Sandoz Pharamaceutical Inc.); treated with the costimulation blockade plus rapamycin (Wyeth Research Institute, Princeton, N.J.); treated with only cyclosporine; treated with only rapamycin; and untreated. A control group of three C3H/He mice received syngeneic transplants. The treatments were given as follows:

Group 1) Anti-CD40L+CTLA4-Ig

Anti-CD40L: 250 μg/injection i.v. (intravenous) on day 0, i.p. (intraperitoneal) on day +2, +4,

CTLA4-Ig: 200 μg/injection i.p. on day 0, +2, +4, +6.

Group 2) Anti-CD40L+CTLA4-Ig+CsA

Anti-CD40L: 250 μg/injection i.v. on day 0, i.p. on day +2, +4,

CTLA4-Ig: 200 μg/injection i.p. on day 0, +2, +4, +6,

CsA: 20 mg/kg/day s.c. (subcutaneous) every day from day 0 to +14.

Group 3) Anti-CD40L+CTLA4-Ig+RPM

Anti-CD40L: 250 μg/injection i.v. on day 0, i.p. on day +2, +4,

CTLA4-Ig: 200 μg/injection i.p. on day 0, +2, +4, +6

RPM: 0.2 mg/kg/day i.p. on day 0, +1, +2, then every other day to day +14.

Group 4) CsA

CsA: 20 mg/kg/day s.c. every day from day 0 to day +14.

Group 5) RPM

RPM: 0.2 mg/kg/day i.p. on day 0, +1, +2, then every other day to day +14. The hosts were checked daily for graft survival by palpitation. In addition, other mice were sacrificed for pathologic examination at various time points.

### Results

The results are shown in the FIGURE and the Table. All of the untreated mice receiving the allografts died by day 11. The cyclosporine treatment alone prolonged survival, but all mice died between day 13 and day 27. The allograft recipients receiving the addition of the costimulation blockade to the cyclosporine treatment extended survival for a longer period of time, but all mice in this group died by day 41. In contrast, mice which received solely the costimulation blockade, solely rapamycin, or both rapamycin and the costimulation blockade all survived, as did the control mice.

Effects of CSA, RPM and Costimulation Blockade Treatment on Graft Survival.

| Treatment | Graft Survival |
|---|---|
| Untreated | 8, 9, 10, 10, 11 |
| MR1/CTLA4-Ig | >100, >73, >73, >70, >70 |
| MR1/CTLA4-Ig/CsA | 25, 26, 29, 35, 41 |
| MR1/CTLA4-Ig/RPM | >100, >100, >100, >91, >91 |
| CsA | 13, 19, 19, 27 |
| RPM | >100, >100, >100, >97, >89 |
| Syngeneic | >35, >35, >35 |

MR1 = anti-CD40L; CsA = cyclosporine A; RPM = rapamycin.

Equivalents

While this invention has been particularly shown and described with references to preferred embodiments thereof, it will be understood by those skilled in the art that various changes in form and details may be made therein without departing from the spirit and scope of the invention as defined by the appended claims. For example, those skilled in the art will recognize or be able to ascertain using no more than routine experimentation, many equivalents and substantial equivalents to the specific embodiments of the invention described specifically herein. Such equivalents and substantial equivalents are intended to be included in the scope of the claims.

What is claimed is:

1. A method of inducing transplant tolerance in a mammal, comprising inhibiting T-cell costimulation and IL-2 mediated T-cell proliferation without inhibiting IL-2 mediated T-cell apoptosis.

2. A method of inducing transplant tolerance in a mammal, comprising administering to said mammal an effective dose of a T-cell costimulation blockade agent and an effective dose of an immunosuppressive agent, while maintaining normal levels of T-cell death.

3. The method of claim 2 wherein the immunosuppressive agent is rapamycin, or a biologically active derivative thereof.

4. The method of claim 2 wherein the costimulation blockade agent and the immunosuppressive agent are administered substantially simultaneously.

5. The method of claim 2 wherein the costimulation blockade agent and the immunosuppressive agent are administered simultaneously and subsequently the immunosuppressive agent is administered continuously at effective doses.

6. The method of claim 2 wherein the costimulation blockade agent comprises at least one agent that blocks a signaling pathway mediated by CD40, CD40L, B7, CD28 or CTLA4.

7. The method of claim 6 wherein the costimulation blockade agent comprises at least one agent selected from the group consisting of anti-CD40 antibodies, anti-CD40L antibodies, anti-B7 antibodies, anti-CD28 antibodies, anti-CTLA4 antibodies, B7-Ig, CD28-Ig CD40-Ig, CD40L-Ig, CTLA4-Ig, soluble extracellular domain proteins of CD40, CD40L, B7, CD28 and CTLA4 and derivatives thereof, and costimulation blockade drugs.

8. The method of claim 2 wherein the costimulation blockade agent comprises anti-CD40L and CTLA4-Ig.

9. The method of claim 3 wherein the rapamycin is contained in a fish oil composition.

10. The method of claim 3 wherein the route of administration is intraperitoneal, intravenous, oral or subcutaneous.

11. A composition comprising at least one costimulation blockade agent and rapamycin, or a biologically active derivative thereof.

12. The composition of claim 11 further comprising fish oil.

13. The composition of claim 11 wherein the costimulation blockade agent comprises at least one agent selected from the group consisting of anti-CD40 antibodies, anti-CD40L antibodies, anti-B7 antibodies, anti-CD28 antibodies, anti-CTLA4 antibodies, B7-Ig, CD28-Ig, CD40-Ig, CD40L-Ig, CTLA4-Ig; soluble extracellular domain proteins of CD40, CD40L, B7, CD28 and CTLA4 and derivatives thereof, and costimulation blockade drugs.

14. A kit comprising at least one costimulation blockade agent and rapamycin.

15. A method of inducing transplant tolerance in a mammal comprising administering a T-cell costimulation blockade agent, wherein T-cell proliferation, but not T-cell apoptosis is inhibited.

16. A method of inhibiting T-lymphocyte induced rejection of an allograft in a mammal comprising administering a T-cell costimulation blockade agent and an immunosuppressive agent, wherein T-cell costimulation and IL-2 mediated T-cell proliferation are inhibited and IL-2 mediated T-cell apoptosis is not inhibited.

17. The method of claim 16 further comprising administering an effective dose of rapamycin or a derivative thereof.

18. A method of prolonging the survival of an allograft in a mammal, comprising administering a T-cell costimulation blockade agent and an immunosuppressive agent, wherein T-cell costimulation and IL-2 mediated T-cell proliferation are inhibited and IL-2 mediated T-cell apoptosis is not inhibited.

19. The method of claim 18 further comprising administering an effective dose of rapamycin or a derivative thereof.

20. A method for inducing T-cell non-responsiveness to a donor tissue or organ in a recipient, comprising administering a T-cell costimulation blockade agent and an immunosuppressive agent, wherein T-cell costimulation and IL-2 mediated T-cell proliferation are inhibited and IL-2 mediated T-cell apoptosis is not inhibited.

21. The method of claim 20 further comprising administering an effective dose of rapamycin or a derivative thereof.
